(19) United States
(12) Patent Application Publication
(10) Pub. No.: US 2017/0095442 A1
(43) Pub. Date: Apr. 6, 2017

(54) Methods of Treating Salmonella-Induced Diarrhea in Non-Human Animals

(71) Applicant: JAGUAR ANIMAL HEALTH, INC., San Francisco, CA (US)

(72) Inventor: Serge Martinod, Groton, CT (US)

(73) Assignee: Jaguar Animal Health, Inc., San Francisco, CA (US)

(21) Appl. No.: 15/314,175

(22) PCT Filed: May 28, 2015
(86) PCT No.: PCT/US2015/032927

§ 371 Date: Nov. 28, 2016

Related U.S. Application Data
(60) Provisional application No. 62/004,657, filed on May 29, 2014.
(60) Provisional application No. 62/087,210, filed on Dec. 3, 2014.

(51) Int. Cl. A61K 31/353; A61K 36/47; A61K 9/51; A23K 10/30; A61K 45/06; A23K 50/20; A23K 50/10; A23K 50/60; A61K 9/00; A61K 9/06

(52) U.S. Cl. CPC A61K 31/353; A61K 9/0068; A61K 36/47; A61K 9/0095; A61K 9/51; A61K 9/06; A61K 45/06; A61K 9/006; A23K 50/20; A23K 50/10; A23K 50/60; A23K 10/30

(57) ABSTRACT

Provided are methods of treating diarrhea, particularly malabsorption diarrhea, in neonatal, young and adult non-human animals, in which the diarrhea results from infection of the animals by Salmonella spp., such as Salmonella typhimurium, with a therapeutically effective amount of a proanthocyanidin polymer from Croton lechleri, in either enteric or non-enteric form.

### FIELD OF THE INVENTION

The invention relates to the treatment of Salmonella spp.-induced diarrhea in non-human animals involving the use of enteric or non-enteric formulations of a proanthocyandin polymeric composition isolated from the plant Croton spp. or Calophyllum spp., or with a latex, extract, or food supplement derived therefrom. More particularly, the composition is effective in treating secretory or watery diarrhea caused by Salmonella spp. infection, reducing the severity and duration of diarrhea in non-human animals, as well as increasing their survivability.

### BACKGROUND OF THE INVENTION

A wide array of infectious and pathogenic agents including bacteria, viruses, and parasites cause diarrhea in non-human animals, particularly, domesticated livestock animals associated with farming, food, and labor. Many of these enteropathogens cause one or more adverse effects in the animals, such as severe intestinal lesions, dehydration, alterations in enzyme activity, and/or alterations in nutrient transport mechanisms. The clinical presentation of diarrhea caused by these agents may vary; some diarrheas are self-limiting, while others are associated with high morbidity or high mortality (R. E. Holland, 1990, Clin. Microbiol. Rev., 3(4):345-375).

Secretory diarrheas, also called watery diarrheas, are a major source of illness and mortality in both young and adult non-human animals and are characterized by the loss of both fluid and electrolytes through the intestinal tract, leading to serious and often life-threatening dehydration. Secretory diarrhea is caused by a variety of bacterial, viral and protozoal pathogens and also results from other non-infectious etiologies such as ulcerative colitis, inflammatory bowel disease, environmental and stress conditions, and cancers and neoplasias of the gastrointestinal tract. All types of diarrheal disease may have a secretory component.

Salmonellosis is an infection with Salmonella spp. are bacterial microorganisms that can cause secretory diarrhea in non-human animals. Salmonellosis is rising in prevalence in the cattle industry and is most common in dairy calves one to ten weeks of age, but can also be seen in adult dairy cows and beef cattle. Salmonellosis has a serious economic impact on the cattle industry worldwide. Livestock mortality, treatment costs, abortion, reduced production, discarded milk and reduced consumer confidence all contribute to the cost to cattle industries of disease caused by Salmonella. Fecal contamination of feed and water from shedding cattle to naïve cattle is a common source and route of infection. Contaminated milk, contaminated processed feeds, and improperly cleaned calf-feeding equipment can also serve as sources of infection. Ravens, opossums, pigeons, rats and mice can also serve as carriers or vectors.

Salmonellosis is also a significant public health concern. Humans and other animals can become infected from consumption of contaminated drinking water, raw dairy and milk products, and undercooked meat products.

The available and commonly used treatments for Salmonella-induced diarrhea in non-human animals typically involve vital fluid replacement and electrolyte replenishment to counter or stop fluid and electrolyte loss. Other types of treatments include the administration of oral antibiotics and non-steroidal anti-inflammatory drugs. Depending on the cause(s), timing, severity and course of diarrhea and/or its associated disease or condition, the various known treatments may or may not be effective, and the animals may or may not respond adequately or in a timely manner, leading to premature death in many cases. In addition, the use of antibiotics for Salmonella enteritis without septicemia (bacteremia) is controversial because the population of normal intestinal bacterial microflora in the animal's gut may be altered, and the potential for developing antibiotic resistant Salmonella organisms exists.

Because the economic and humane impacts of diarrhea and its related conditions on the afflicted animals, their handlers and caregivers are so great, there is a compelling need for alternative, safe, and medically effective, as well as cost effective, treatments and remedies for diarrhea and its associated symptoms in non-human adult animals. The present invention addresses such a need.

### SUMMARY OF THE INVENTION

The present invention provides a method of treating diarrhea, such as malabsorption diarrhea, resulting from Salmonella spp. infection of a non-human animal, in which the method involves orally administering to an animal in need thereof a pharmaceutically acceptable composition comprising an aqueous soluble proanthocyanidin from Croton lechleri, wherein the composition is formulated in an effective amount to treat the Salmonella-induced diarrhea in the animal. In an aspect, the methods effectively treat the symptoms associated with the Salmonella-induced diarrhea in afflicted animals; the symptoms may include dehydration, weight loss and electrolyte loss. In an embodiment, the diarrhea is episodic.

In embodiments of the above method, the pharmaceutically acceptable C. lechleri proanthocyanidin composition is an enteric formulation. In other embodiment, the pharmaceutically acceptable C. lechleri proanthocyanidin composition is an non-enteric formulation. In embodiments of the method, the C. lechleri proanthocyanidin polymer composition is SB 300, SP 303, or crofelemer. In an embodiment, the non-human animal is a non-adult, e.g., neonatal or young animal, or an adult animal. The method of the invention is not particularly limited as to the species of the animal and can include, exotic, zoo, farm, or domestic animals, e.g., without limitation, bovine, equine, ovine, swine animals, goats, bison, buffalo, or camels. In particular embodiments, the non-human animal is an equine or bovine animal, or a camel.

In other embodiments of the above method the C. lechleri proanthocyanidin polymer or a composition comprising the C. lechleri proanthocyanidin polymer is administered to the animal in an amount of at least 50 mg to 250 mg. In an embodiment, the C. lechleri proanthocyanidin polymer, or a composition comprising the C. lechleri proanthocyanidin polymer, is administered to the animal in an amount of at least 250 mg twice daily. In still other embodiments of the method, the C. lechleri proanthocyanidin polymer composition is administered as powder reconstituted with a liquid selected from oral electrolytes, milk, milk replacer, physiological saline, or water. In other embodiments of the method, the C. lechleri proanthocyanidin polymer composition is administered as a bolus, such as a pill, tablet or capsule. In an embodiment, the C. lechleri proanthocyanidin polymer composition is orally administered. In addition, the C. lechleri proanthocyanidin polymer composition may be administered in animal feed or drink. In other embodiments of the method, the non-human neonatal, young, or adult animal is additionally suffering from an infection or disease associated with one or more of bacteria, parasites, viruses, or protozoa.

In other embodiments of the method, the C. lechleri proanthocyanidin polymer composition is in the form of a gel, paste, or gel paste, which is typically administered to the animal by topical application to the roof of the animal's mouth. In some cases, the gel, paste, or gel paste is contained in a delivery device, which can be a syringe, such as a needle-less syringe. In some embodiments of the method, the gel, paste, or gel paste comprises polymeric microparticles or nanoparticles containing the C. lechleri proanthocyanidin polymer composition. In an embodiment, the polymeric microparticles or nanoparticles are pH-sensitive.

In an embodiment, the methods improve gut health and control diarrhea in the affected animals by providing a proanthocyanidin polymer from Croton lechleri in an effective amount to control or treat the diarrhea in the treated animals. In an embodiment the proanthocyanidin polymer is a formulation, composition, or extract from Croton lechleri. In an embodiment, the proanthocyanidin polymer from Croton lechleri is a more highly purified composition containing proanthocyanidin polymer or oligomer, such as crofelemer or SB 300 compositions described herein. In an embodiment the proanthocyanidin polymer is a formulation, composition, or botanical extract from Croton lechleri. In an embodiment, the formulation, composition, or botanical extract from Croton lechleri is in the form of a paste or gel. In a particular embodiment, the paste formulation comprises beads (nano or microparticles) comprising enterically coated SB 300 or SP 303 and is orally administered to animals in need. In an embodiment, the paste formulation comprises beads (nano or microparticles) comprising enterically coated SB 300. In a particular embodiment, the paste comprising SB 300 enteric beads is orally administered to an animal twice daily for three days. In some embodiments, the paste is orally administered for three consecutive days. In an embodiment, the paste comprising SB 300 enteric beads is orally administered to an animal in need at a dose of 2 mg/kg twice daily for three days.

### BRIEF DESCRIPTION OF THE FIGURES

FIG. 1 shows the effect of a treatment method involving the administration of proanthocyanidin polymer from Croton lechleri according to the invention on average fecal score number as described in Example 1 herein. Salmonella-infected calves were scored twice daily for three days using a 3 level scoring system. As observed, the C. lechleri proanthocyanidin polymer-treated calves demonstrated faster improvement on diarrhea scores starting on the second day after treatment. In FIG. 1, the average fecal score is shown on the y-axis and the number of treatment days is shown on the x-axis.

### DETAILED DESCRIPTION OF THE INVENTION

Salmonellosis affects both young and adult non-human animals, particularly bovine animals in the dairy industry, and seriously affects the economy of the industry, ultimately resulting in livestock death. The most common Salmonella isolates from cattle include Salmonella typhimurium and Salmonella dublin (National Veterinary Services Laboratories, Ames, Iowa; Orient Point, N.Y.). Once ingested, Salmonella bacteria colonize and multiply in the intestine resulting in acute infection. Typical clinical signs of acute salmonella enteritis include fever and severe watery diarrhea with subsequent rapid onset of dehydration. In addition, the bacteria produce toxins that can contribute to gut damage and have systemic effects. If sufficient damage occurs to the intestinal lining, the bacteria may enter the bloodstream, resulting in septicemia, and spread to the brain, lungs, joints, uterus (frequently causing abortion in pregnant bovine animals) and other organs.

Cattle can be chronically infected with Salmonella and serve as carriers within the herd without exhibiting clinical signs. Reports have indicated that one carrier cow can shed one billion salmonellae per day in the feces.

In view of the serious effects of Salmonella infection in non-human animals, the methods of the present invention provide a new means to treat diarrhea resulting from infection of afflicted animals. The methods of the invention provide treatment of diarrhea resulting from infection of non-human animals by the Salmonella spp. microorganism with an effective amount of a polymeric proanthocyanidin composition from a Croton species or Calophyllum species, or with a latex, extract or food supplement botanical extract derived therefrom. The effective treatment of diarrhea caused by Salmonella spp. with a proanthocyanidin polymer composition from Croton lechleri, or with a latex, extract or food supplement botanical extract derived therefrom is an unexpected and surprising aspect of the invention, in large part because Salmonella spp. microorganisms cause diarrhea by a mechanism of action and by affecting cellular pathways and responses that are distinct and different from the mechanism of action typically associated with the activity of proanthocyanidin polymer compositions.

More specifically, the mechanism of action of polymeric proanthocyanidin compositions, or purified extracts such as crofelemer, is through the inhibition of both the cystic fibrosis transmembrane conductance regulator protein (CFTR) chloride ion channel and the calcium-activated chloride ion channels (CaCC). The polymeric proanthocyanidin composition acts by blocking chloride ion channel secretion and the accompanying high volume water loss occurring in diarrhea, thus normalizing the flow of chloride ions and water in the gastrointestinal (GI) tract. However, Salmonella microorganisms trigger diarrhea in infected hosts by producing several virulence factors. One such factor is a protein called SopE, which is injected into intestinal epithelium cells where it triggers a cascade of intracellular signaling events once the bacteria enter the GI tract. (See, e.g., S. Zhang et al., 2003, Infection and Immunity, 71(1):1-12; and A. J. Mueller et al., 2009, Cell Host and Microbe, 6(2):125-136). The binding of the SopE protein to two specific GTPase proteins alters the cell membrane and allows the bacteria to penetrate the cell. In addition, the two GTPase proteins activate Caspase-1 inside the cell, which is a key factor in inflammatory responses. Caspase-1, in turn, causes the production of proinflammatory mediators (cytokines) that attract macrophages which phagocytize the bacteria that has penetrated into the intestinal tissue and cells; however, Salmonella bacteria remaining in the intestinal lumen are not seriously affected. The heightened immune response that exists in the infected animals as a consequence of the infection results in serious inflammation, fluid accumulation and distress for the host animal.

Because Salmonella, which causes a disease pathology and an inflammatory immune response that lead to diarrhea without significantly affecting the CTRF or CaCC, it is considered quite surprising and unexpected that a proanthocyanidin polymer composition which functions by inhibiting these channels is effective in treating diarrhea induced by the Salmonella microorganism. However, the treatment of diarrhea in Salmonella-infected animals, including adult animals and neonatal and young animals, such as, e.g., bovine calves and piglets, with a Croton lechleri proanthocyanidin polymer composition (e.g., SB 300) according to present methods demonstrates an unpredicted effectiveness of the composition against diarrhea resulting from a source associated with a different etiology.

The invention provides methods directed to treating the debilitating problem of Salmonella-induced diarrhea in neonatal, unweaned, young and adult non-human animals. The methods are effective in reducing and/or alleviating Salmonella-induced diarrhea in such non-human animals in need thereof In particular, the methods are directed to the treatment of diarrhea, particularly secretory/watery diarrhea, caused by Salmonella infection, alone or in combination with other infectious agents or environmental conditions, in adult animals and in neonatal and young (juvenile, non-adult) animals, particularly where scourges of diarrhea in such animals can have a profound economic impact for the animal agriculture, food and health industries. The invention further provides formulations and compositions suitable for treating diarrhea in neonatal, young and adult animals. Unless otherwise noted herein, use of the term “animal” herein denotes non-human, warm-blooded mammals of a number of different species. In addition, the terms “young”, non-adult”, “immature” and “juvenile” are used synonymously herein. Without wishing to be limiting, “young” animals are generally under one year of age. “Neonatal” animals are generally two weeks of age or less.

The methods of the invention provide a solution to a significant need for the animal industry, e.g., the beef and dairy industries worldwide, particularly for countries in which neonatal calf diarrhea presents one of the largest health challenges, as well as economic losses.

The methods and treatments of the invention are particularly suitable for treating animals of a young age, as well as adult animals, that have contracted Salmonella infection. In an embodiment, the animals are neonatal (or newborn), unweaned, non-adult animals that are born, bred, raised and/or maintained in a domesticated and/or agricultural setting, e.g., as livestock and farm animals, for commodities such as food, labor, sport, or other commercial or non-commercial agricultural husbandry capacity. In an embodiment, the animals are adult animals in the aforementioned settings. Nonlimiting examples of animals affected by diarrhea and treatable by the methods and formulations of the invention include, without limitation, neonatal and young cattle (calves), pigs (piglets), sheep (lambs), goats (kids), horses (foals) and camels (calves), and adult animals, including, cattle, steer, bison, buffalo, horses, camels, goats, sheep and rams, as further described herein. In an embodiment, the neonatal, young, or adult animals are domestic, companion animals, such as, without limitation, dogs and cats of any species. As used herein, the terms “neonatal” and “newborn” are synonymous. In particular embodiments, animals may be administered the proanthocyanidin polymer compositions of the invention to prevent Salmonella induced diarrhea in an animal or population of animals exposed to or suspected to have been exposed to Salmonella, for example, in connection with a Salmonella outbreak in a herd or other group of animals held in close proximity.

The present invention particularly relates to treating Salmonella-induced diarrhea in adult, or in neonatal, unweaned and young, animals with physiologically and pharmaceutically acceptable formulations and compositions comprising a therapeutically effective amount of an antidiarrheal agent comprising a proanthocyanidin polymer obtained from a Croton spp., preferably Croton lechleri (C. lechleri). The proanthocyanidin polymer composition can also be obtained from a Calophyllum spp., in particular Calophyllum inophylum. In an specific embodiment, the pharmaceutically acceptable composition comprises a proanthocyanidin polymer from Croton lechleri. In a particular embodiment, the proanthocyanidin polymer is enterically protected beads, including enteric beads including SB 300 or SP 303.

In general terms, “treating” an animal according to the present methods refers to achieving or obtaining a desired physiologic and/or pharmacologic effect, whether prophylactic, therapeutic, or both. As used herein “treating” or “treatment” can refer to ameliorating, preventing, inhibiting, reversing, attenuating, alleviating, abrogating, minimizing, suppressing, reducing, decreasing, diminishing, stabilizing, eradicating, curing, or eliminating the deleterious effects of a disease or condition, or the progression or worsening of the disease or condition. For example, successful treatment may involve alleviating one or more symptoms of a disease or condition, although not necessarily all of the symptoms, of the disease or condition, or attenuating the symptoms or progression of the disease or condition. Curing or eliminating the disease or condition from the animal is an optimal outcome of the practice of the methods of the invention.

According to the invention, treatment of an animal in need thereof typically involves the use or administration of an effective amount or a therapeutically effective amount of a proanthocyanidin polymer or a proanthocyanidin polymer composition preferably from a Croton spp. particularly C. lechleri, either enteric or non-enteric. Effective amount refers to the quantity (amount) of the composition, and the like, that induces a desired response in the animal subject upon administration or delivery to the animal. Optimally, an effective amount produces a therapeutic effect in the absence of, or with little or virtually no, adverse effects or cytotoxicity in the animal. Alternatively, any adverse effects associated with an effective amount are optimally outweighed by the therapeutic benefit achieved.

The treatment methods are directed to ameliorating, preventing, inhibiting, reversing, attenuating, alleviating, abrogating, minimizing, suppressing, reducing, decreasing, diminishing, stabilizing, eradicating, curing, or eliminating diarrhea and/or its associated symptoms caused by Salmonella spp. infection that adversely affect the health, growth and survivability of neonatal and young, as well as adult, animals. In an embodiment, the diarrhea is secretory/watery diarrhea. Such diarrhea can be a clinical sign of gastrointestinal (GI) disease in an animal; it can also reflect primary disorders outside of the digestive system, such as disorders affecting the large bowel or the small bowel. The methods described herein are also suitable for treating diarrhea resulting from Salmonella infection in animals that may have comorbid conditions or disorders that cause diarrhea via different mechanisms involved in their pathogenesis, for example, osmotic diarrhea, secretory diarrhea, episodic diarrhea, or inflammatory and infectious diarrhea. In an embodiment, the neonatal or young animal can suffer from diarrhea associated with inflammation of the lining of the colon, such as colitis, or acute colitis, which can be caused by infection or inflammation of the bowel.

Osmotic diarrhea is associated with absorption of water in the intestines, which depends upon adequate absorption of solutes. If excessive amounts of solutes are retained in the intestinal lumen, water will not be absorbed and diarrhea results. Osmotic diarrhea typically results from ingestion of a poorly absorbed substrate, for example, a carbohydrate or divalent ion or from malabsorption of any type, such as an inability to absorb certain carbohydrates. Secretory diarrhea occurs when the secretion of water into the lumen of the intestine exceed absorption. Under normal conditions, large volumes of water are secreted into the small intestinal lumen, but a large portion of this water is efficiently absorbed before reaching the large intestine.

Secretory diarrhea can result from exposure of an animal to toxins (enterotoxins) from certain types of bacteria, such as cholera toxin of Vibrio cholerae and heat-labile toxin of E. coli. Massive diarrhea is induced from such microorganisms as a consequence of their toxins strongly activating adenylyl cyclase, which causes a prolonged increase in the intracellular concentration of cyclic AMP within crypt enterocytes. This increase. in turn. results in prolonged opening of the chloride channels that contributes to secretion of water from the crypts, thereby allowing uncontrolled secretion of water. These bacterial toxins can also affect the enteric nervous system, resulting in an independent stimulus of water secretion.

Inflammatory and infectious diarrhea can be caused by the disruption of the epithelium of the intestine due to microbial or viral pathogens. Typically, the epithelium of the digestive tube is protected from insult by a number of mechanisms that constitute the gastrointestinal barrier. However, the gastrointestinal barrier can be breached and result in diarrhea. Destruction of the epithelium results not only in leaking of serum and blood into the lumen but also is often associated with significant destruction of adsorptive epithelium. When this occurs, the absorption of water becomes highly inefficient and diarrhea results. The pathogenic culprits frequently associated with infectious diarrhea include bacteria, such as E. coli, Campylobacter and Salmonella; viruses, such as rotaviruses, coronaviruses, parvoviruses and norovirus; and protozoa, such as coccidia species, Cryptosporium and Giardia. In addition, the response of the immune system to inflammatory conditions in the bowel contributes greatly to the development of diarrhea. Activated white blood cells are stimulated to produce and secrete inflammatory mediators and cytokines that stimulate secretion. An secretory component is thus imposed upon and exacerbates an inflammatory diarrhea. Moreover, reactive oxygen species produced by leukocytes can damage or destroy intestinal epithelial cells, which are replaced with immature cells that are generally lacking in the brush border enzymes and transporters necessary for the absorption of nutrients and water. Thus, components of an osmotic (malabsorption) diarrhea provide additional pathology and problems for an afflicted animal.

Thus, in a particular embodiment, the diarrhea to be treated is caused by infection or invasion of the animals by Salmonella spp. pathogens. In other embodiments, the animal may suffer from both Salmonella-induced diarrhea and diarrhea resulting from one or more non-Salmonella spp. infectious agents, including bacteria, e.g., Escherichia coli, Clostridium perfringes, etc.; viruses, e.g., coronaviruses, rotaviruses, bovine virus diarrhea (BVD) virus, infectious bovine rhinotracheitis (IBR) virus, etc.; protozoa, e.g., Cryptosporidium, coccidia, etc.; as well as yeasts and molds. The methods and compositions of the invention are suitable for treating animals afflicted with such types of mixed infections.

The treatment of Salmonella-induced diarrhea in neonatal and young animals according to the methods of the invention is of particular importance, because such immature animals are most susceptible to infection by Salmonella spp., as well as numerous other types of pathogens, and resistance to infection develops with increasing age of the animal. In addition, younger animals experience more severe clinical illness as a result of infection and resulting diarrhea. In general, due to the anatomy of the gastrointestinal tract of adult animals such as horses, conditions affecting the large intestine and cecum typically cause diarrhea. However, young animals, e.g., foals, that are less than about three months of age do not have fully competent large intestines and ceca as do adult animals; therefore, young animals tend to be more prone to diarrhea caused by small intestinal conditions. In general terms, a foal is an equine, particularly a horse, that is one year old or younger in age.

In some cases, in addition to Salmonella-induced diarrheal disease, noninfectious causes can augment or worsen the problems experienced by the animals, for example, without limitation, inadequate nutrition and/or insufficient attention of a neonate or young animal on the part of the mother, or in both young and adult animals, exposure to severe environment, or a combination of these events. In another embodiment, diarrhea results from a combination of the invasion of infectious Salmonella spp. and noninfectious factors. Frequently, noninfectious causes of diarrhea in young or adult animals are considered to be factors that predispose or contribute to an animal's susceptibility to infectious agents and causes of diarrhea. Whether the cause of diarrhea in animals is infectious or noninfectious, the absorption of fluids from the intestine is altered and life-threatening electrolyte imbalances can occur. The affected animals lose fluids, rapidly dehydrate and suffer from electrolyte loss and acidosis. Although infectious agents may cause an initial damage to the animal's intestine, actual death from diarrhea (serious diarrhea) in animals usually is a consequence of dehydration, acidosis and loss of electrolytes, which may be difficult to replenish in adequate amount and time. Accordingly, the methods and formulations of the invention are suitable for treating diarrhea and the symptoms of diarrhea, such as dehydration and electrolyte loss, in an effort to prevent more severe dehydration and animal death.

Proanthocyanidins and Tannins Obtained from Plant Extracts

Proanthocyanidins are types of condensed tannins, which are found in a large number of plants and are classified as hydrolyzable or condensed. Tannins and, in particular, proanthocyanidins are contained in many plants used in traditional medicine as treatment or prophylaxis for diarrhea (See, e.g., Yoshida et al., 1993, Phytochemistry, 32:1033; Yoshida et al., 1992, Chem. Pharm. Bull., 40:1997; Tamaka et al., 1992, Chem. Pharm. Bull., 40:2092).

Proanthocyanidins are comprised of at least two or more monomer units that may be of the same or different monomeric structure. The monomer units (generally termed “leucoanthocyanidins”) are generally monomeric flavonoids which include catechins, epicatechins, gallocatechins, galloepicatechins, flavanols, flavonols, flavan-3,4-diols, leucocyanidins and anthocyanidins. The polymer chains are thus based on different structural units, creating a wide variation of polymeric proanthocyanidins and a large number of possible isomers (Hemingway et al., 1982, J. C. S. Perkin, 1:1217). Larger polymers of the flavonoid 3-ol units are predominant in most plants and often have average molecular weights above 2,000 daltons (Da), containing 6 or more units (Newman et al., 1987, Mag. Res. Chem., 25:118).

Proanthocyanidin polymers and proanthocyanidin are found in a wide variety of plants, especially those having a woody habit of growth (e.g., Croton spp. and Calophyllum spp.). A number of different Croton tree species, including Croton sakutaris, Croton gossypifolius, Croton palanostima, Croton lechleri, Croton erythrochilus and Croton draconoides, which are endemic to South America, produce a red viscous latex sap called Sangre de Drago or “Dragon's Blood”. The red viscous latex is known for its medicinal properties. For example, U.S. Pat. No. 5,211,944 describes the isolation of an aqueous soluble proanthocyanidin polymer composition from Croton spp. (See also, Ubillas et al., 1994, Phytomedicine, 1:77). The isolation of an aqueous soluble proanthocyanidin polymer composition from Calophyllum inophylum in U.S. Pat. No. 5,211,944.

In an embodiment, a proanthocyanidin polymer from C. lechleri, or a composition thereof, is crofelemer. Crofelemer (CAS 148465-45-6) is an oligomeric proanthocyanidin of varying chain lengths derived from the Dragon's Blood of Croton lechleri, a tree of the family Euphorbiaceae, which is sustainably harvested under fair trade work practices in the Amazon. It has an average molecular weight of approximately 1900 Da to approximately 2700 Da. The monomers comprising crofelemer comprise catechin, epicatechin, gallocatechin, and epigallocatechin. The chain length of crofelemer ranges from about 3 to about 30 units with an average chain length of about 8 units. Crofelemer has the chemical formula: (C₁₅O_{6,7}H₁₂)ₙ and a molecular mass of 860-9100 g/mol. The antisecretory mechanism of action of crofelemer involves the targeting and inhibition of two, distinct intestinal chloride channels, namely, the cystic fibrosis transmembrane regulator conductance (CFTR) channel, which is a cAMP-stimulated Cl⁻ channel, and the calcium-activated chloride channel (CaCC), as reported, for example, by Tradtrantip, L. et al., 2010, “Crofelemer, an Antisecretory Antidiarrheal Proanthocyanidin Oligomer Extracted from Croton lechleri, Targets Two Distinct Intestinal Chloride Channels”, Mol. Pharmacol., 77(1):69-78). A general structure of crofelemer is shown below. In the structure, an H at the R position of the structure signifies procyanidin; an OH at the R position of the structure signifies prodelphinidin.

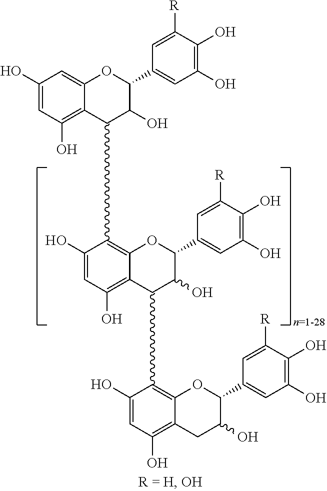

In accordance with an embodiment of the invention, crofelemer, or a pharmaceutically acceptable formulation or composition comprising crofelemer, is employed in the treatment methods as the proanthocyanidin polymer from Croton lechleri.

In an embodiment, SP 303, an oligomeric proanthocyanidin from Croton lechleri, (also known as crofelemer) is the proanthocyanidin polymer from Croton lechleri, or a pharmaceutically acceptable formulation or composition comprising SP 303, which is suitable for use in the treatment methods of the invention. SP-303 (R. Ubillas et al., 1994, Phytomedicine, 1:77-106) is largely composed of purified proanthocyanidin oligomers (−)-galloepicatechin and (+)-gallocatechin,(−)-epicatechin and (+)-catechin and is suitable for use in the enteric and non-enteric formulations and compositions for administration in the treatment methods described herein.

In another embodiment, SB 300, a proanthocyanidin polymer extract from Croton lechleri is the proanthocyanidin polymer from Croton lechleri, or a pharmaceutically acceptable formulation or composition comprising SB 300, which is suitable for use in the treatment methods of the invention. SB 300, as described, for example, by Fischer, H. et al., (2004, J. Ethnopharmacol., 93(2-3):351-357) provides a natural product extract that is particularly amenable for both enteric and non-enteric formulations and compositions, and is highly functional and cost-effective in the treatment methods described herein.

A pharmaceutically acceptable composition comprising a proanthocyanidin polymer from Croton lechleri and employed in the treatment methods of the invention can be obtained from C. lechleri, e.g., as described in WO 00/47062 to Shaman Pharmaceuticals, Inc., the contents of which are incorporated herein, and formulated as a food or dietary supplement or nutraceutical formulation.

In other embodiments, compositions useful in the methods of the invention comprise a raw latex obtained from a Croton species or a Calophyllum species, or an extract obtained from a Croton species or a Calophyllum species, which are not specifically polymeric proanthocyanidin polymer compositions. Exemplary extracts are described in Persinos et al., 1979, J. Pharma. Sci., 68:124 and Sethi, 1977, Canadian J. Pharm. Sci., 12:7.

In an embodiment, the proanthocyanidin polymer from Croton lechleri is formulated with an enteric coating or matrix in a variety of dosage formats known in the art (See, e.g., WO 00/47062 and U.S. Pat. Nos. 7,441,744 and 7,323,195, the contents of which are incorporated herein, and as briefly described below. In another embodiment, the proanthocyanidin polymer is formulation without an enteric coating or matrix. Both enteric and non-enteric forms of the proanthocyanidin polymer from Croton lechleri, for example, SB 300, are intended for use in the methods of the present invention.

### Preparation of Proanthocyanidin Polymer Compositions and Formulations

The proanthocyanidin polymer composition, effective for treating secretory diarrhea according to the invention, is comprised of monomeric units of leucoanthocyanidins. More particularly, the composition is comprised of proanthocyanidin polymers of 2 to 30 flavonoid units, preferably 2 to 15 flavonoid units, more preferably 2 to 11 flavonoid units and most preferably an average of 7 to 8 flavonoid units with a number average molecular weight of approximately 2500 Da. The proanthocyanidin polymer composition is preferably soluble in an aqueous solution. Preferred for use in the methods according to the invention is a proanthocyanidin polymer from C. lechleri; such a C. lechleri proanthocyanidin polymer may be in the form of a pharmaceutically acceptable composition.

Examples of proanthocyanidin polymeric compositions useful in the present invention are preferably isolated or purified from a Croton spp., namely, Croton lechleri, or Calophyllum spp. by any method known in the art. For example, the proanthocyanidin polymer composition may be isolated from a Croton spp. or Calophylum spp. by the method disclosed in U.S. Pat. No. 5,211,944 or in Ubillas et al. (1994, Phytomedicine, 1:77-106, called SP 303 therein), both of which are incorporated herein by reference. Other isolation methods are described in U.S. Pat. Nos. 7,556,831 and 8,067,041 (Example 2), the contents of which are incorporated herein. PCT application PCT/US00/02687, published as WO 00/47062, the contents of which are incorporated herein, also discloses a method of manufacturing a proanthocyanidin polymeric composition isolated from Croton spp. or Calophyllum spp. and enteric formulations of proanthocyanidin polymer dietary supplements, as well as methods of their preparation. Another illustrative method for isolating proanthocyanidin polymer from C. lechleri (such as crofelemer) is found in U.S. Pat. Nos. 7,341,744 and 7,323,195, the contents of which are expressly incorporated herein. As described above, the SP 303 and SB 300 purified forms of oligomeric proanthocyanidin polymer from Croton lechleri are suitable for use in the treatment methods of the invention.

In an embodiment, the proanthocyanidin polymer composition may be generally isolated by the following process, such as provided in U.S. Pat. No. 7,341,744, the contents of which are incorporated herein. Latex collected from Croton lechleri plants is mixed with purified water (preferably one part latex to two parts purified water). Any insoluble material in the latex solution is allowed to settle, e.g., by leaving the mixture at 4° C. overnight (12 hours). The supernatant is pumped away from the residue and is extracted with a short chain alcohol, such as n-butanol. The extraction is preferably performed multiple times, such as three times. After each extraction, the alcohol phase is discarded and the aqueous phase is retained. The aqueous phase is concentrated, for example, using an ultrafiltration device with a 1 kD cut-off membrane. This membrane can be a low protein binding cellulose membrane, or, alternatively, a polypropylene, teflon or nylon membrane can be used. The membrane used should be compatible with acetone. The purpose of the ultrafiltration is to remove the water from the material.

The retentate from the ultrafiltration is then concentrated to dryness, for example using tray-dryers at approximately 37° C. (±2° C.). The dried material is subsequently dissolved in water and is then chromatographed on a cation exchange column (e.g., a CM-Sepharose column) and a size exclusion column (e.g., an LH-20 column). In the preferred two column system, material is run over a CM-Sepharose and then an LH-20 column in a series. Specifically, the dissolved material is loaded onto the cation exchange column and is then washed with purified water. The proanthocyanidin polymer material is eluted from the cation exchange column with an aqueous acetone solution (preferably 30% acetone), thereby loading the proanthocyanidin polymer material onto the sizing column. The sizing column is disconnected from the cation exchange column and the material is then eluted off of the sizing column with an aqueous acetone solution (preferably 45% acetone). The fractions are collected and monitored with a UV detector, e.g., at a wavelength of 460 nm. Fractions containing the proanthocyanidin polymer material are combined and concentrated, for example, by ultrafiltration using, e.g., a 1 kD cut-off membrane (as described above for the ultrafiltration step prior to the chromatography steps). The retentate may then be concentrated to dryness using a suitable drying method, such as, but not limited to, a rotary evaporator, at a temperature of approximately 37° C. (±2° C.). Other suitable drying methodologies include, but are not limited to, tray drying and spray drying. Example 10 of U.S. Pat. No. 7,341,744 provides additional, non-limiting, methodology for preparing a composition comprising proanthocyanidin polymer, which can be used according to the invention. A detailed protocol for isolating an enriched proanthocyanidin polymer extract suitable for use in the methods of the invention is described in WO 00/47062 as noted herein above.

### Methods of Treatment and Applications of Use

The invention is directed to methods of treating diarrhea associated with pathogenic infection of neonatal, young and adult animals by Salmonella spp. microorganisms. The methods also embrace the treatment of diarrhea induced by Salmonella spp. in combination with non-pathogenic causes in neonatal and young animals, and in adult animals, in need thereof, comprising administering to an animal in need of such treatment, a pharmaceutically acceptable composition comprising a proanthocyanidin polymer from a Croton species or Calophyllum species in an amount effective to treat the diarrhea. In preferred embodiments, the proanthocyanidin polymer is from a Croton species, namely, Croton lechleri. Treating the diarrhea can involve reducing the severity and duration of the diarrhea in the animal. Treating the diarrhea can also involve increasing the survivability of the animal undergoing treatment. In an embodiment, the diarrhea is secretory or watery diarrhea. Treating the Salmonella-infected animals with the proanthocyanidin polymer from Croton lechleri results in treating the diarrhea resulting from the infection, as well as in improving overall health of the animals so that their natural immune function can assist in eradicating the bacterial infection, thereby reducing morbidity and mortality. The invention also provides methods of preventing, including reducing the incidence or severity of, diarrhea in non-human animals, particularly prior to being exposed to events or an environment that might increase the risk of diarrheal disease.

The methods of the invention further include methods of preventing or reducing the incidence or severity of diarrhea in neonatal, young and adult animals having been exposed to or suspected of having been exposed to Salmonella. For example, when one or a few animals in a population is diagnosed with a Salmonella infection, the animals in the herd or other groups of animals that could have come into contact with the infected animal, or its feces or bodily fluids, may be administered a proanthocyanidin polymer composition of the invention to prevent diarrheal disease or at least reduce the incidence or severity of the disease or the symptoms thereof.

In an embodiment of the invention, the methods directed to treating non-human animals, including adult animals, neonates and young animals infected with Salmonella microorganisms (or exposed to or suspected of having been exposed to Salmonella). In embodiments, the methods are directed to treating Salmonella-infected newborns and young of livestock, domestic and farm animals, including grazing animals, which are oftentimes relatively large in size. In one embodiment, the immature animals to which treatment with the proanthocyanidin polymer from Croton lechleri is administered are neonatal (newborn) or infant animals, for example, one to ten hours after birth, one to fifteen hours after birth, twelve to twenty-four hours after birth, twenty-four to thirty-six hours after birth, one to three days after birth, one to four days after birth, one to six days after birth, or one to seven days after birth or up to two weeks after birth. Neonatal animals generally being those under two weeks of age. In an embodiment, the animals are treated between day one and day four after birth. In some embodiments, the neonatal or young animals are treated one to five days of age, less than one week of age, or only a few weeks of age. In an embodiment, treatment occurs during the first weeks of life, for example, one to six weeks of age. In an embodiment, the animals are from two to ten weeks of age, for example, less than one, two, three, four, five, six, seven, eight, nine, or ten weeks of age. The animals undergoing treatment may also be from one to four weeks of age, from one to six weeks of age, or from two to four weeks of age. In some embodiments, the animals are one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, twenty-five, twenty-six, twenty-seven, twenty-eight, twenty-nine, or thirty days old. In other embodiments, the animals are thirty to forty days old. In other embodiments, the animals are young animals, generally up to one year in age. In many cases, the animals are not weaned (unweaned), i.e., they are still drinking milk. For example, dairy calves are generally weaned at 60 to 80 days while beef cattle may be weaned at 3 to 8 months of age, pigs at 3 weeks of age, dogs at 7 to 8 weeks, and horses at 4 to 6 months of age. Also in many cases, neonatal is synonymous with unweaned. In some cases, the animals are newly weaned or weaned, but still juvenile, young, and non-adult. Such young animals are also highly susceptible to becoming afflicted with diarrhea from Salmonella spp., as well as other infectious agents and/or environmental causes.

According to the methods of the invention, the Salmonella-infected neonatal and young animals can be treated with a proanthocyanidin polymer from C. lechleri, e.g., SB 300, or a botanical extract derived from C. lechleri, for one, two, three, four, five, six, seven, eight, nine, or ten days, etc. The C. lechleri proanthocyanidin polymer can be administered to the animal on consecutive days or intermittently, such as every other day, every two days, every three days, every four days, and the like. In an embodiment, the C. lechleri proanthocyanidin polymer is administered to the animals for three consecutive days. In an embodiment, the C. lechleri proanthocyanidin polymer is administered to neonatal animals between one and four days after birth for three consecutive days. As understood by the skilled practitioner, environmental, e.g., farm, conditions surrounding the neonatal and young animals may dictate the start and course of a treatment regimen such that the administration of the C. lechleri proanthocyanidin polymer occurs earlier in the animal's life and for a longer duration, especially since diarrheal disease typically affects neonatal and young animals in about the first seven days of life, or between about day one or day four of life. In the foregoing embodiments, the animals are bovine or camel calves.

The methods of the invention further relate to the treatment of Salmonella-infected (or those exposed to or suspected of having been exposed to Salmonella), adult, non-human animals, such as, without limitation, adult livestock, farm animals, and domestically or commercially used animals. Often, but not always, the animals are large in size and have complex gastrointestinal systems. For example, in horses, which are optimally suited for the diarrhea treatment methods of the invention, the intestinal volume of the animal is large, with the main site of diarrhea being in the colon. Consequently, the described methods provide treatment of such Salmonella-infected animals with a proanthocyanidin polymer from C. lechleri, or composition thereof, providing for an adequate amount and appropriate distribution of the proanthocyanidin polymer in the gut of the animal so as to treat the diarrhea and/or its symptoms and optimally cure the diarrhea in the animal. In a related manner, ruminant animals, such as camels, which are also optimally suited for the diarrhea treatment methods of the invention, possess multi-chambered stomachs, e.g. four stomach compartments, including a rumen or first compartment of the alimentary canal, which serves as the primary site for microbial fermentation of ingested feed. The described methods provide treatment of ruminant animals such as camels, cows, sheep and goats with a proanthocyanidin polymer from C. lechleri, or composition thereof, providing for an adequate amount and appropriate distribution of the proanthocyanidin polymer in the intestine/gut of the animal, rather than the rumen, so as to treat the diarrhea and/or its symptoms and optimally cure the diarrhea.

In an embodiment of the diarrhea treatment methods of the invention, the proanthocyanidin polymer from C. lechleri is orally administered to Salmonella-infected adult animals (or those exposed or suspected to have been exposed to Salmonella) having a large intestinal volume and/or multi-compartment stomachs and a rumen in a large volume and/or high concentration to treat the diarrhea. For example, the C. lechleri proanthocyanidin polymer, or composition thereof, is orally provided or administered to the animal in a volume of about 10 to 50 liters, or in a volume of 20 to 40 liters or in a volume of about 25 to 30 liters, so as to target and reach the animal's large intestine/gut for optimal treatment. In an embodiment in which ruminant animals are treated for diarrhea in accordance with the methods of the invention, the C. lechleri proanthocyanidin polymer, or composition thereof, is formulated for oral administration to the animal such that the C. lechleri proanthocyanidin polymer bypasses the rumen and reaches the intestine for delivery of the composition to the affected area and to treat the diarrhea more efficiently. In adult ruminants there is a need to bypass the rumen to avoid dilution of the product in a large volume of liquid. The ruminants have specific anatomical structure, called the esophageal groove, in the stomach that closes and forms a tube which enables liquid to bypass the rumen. The closing of this esophageal groove is controlled by neural stimulation from suckling. Thus, bypassing the rumen may be achieved, for example, by using a bottle to administer the product.

In a particular embodiment, a formulation or composition comprising a botanical extract derived from C. lechleri, SB 300, or SP 303, is provided in the form of a gel or paste formulation that is orally administered to the young animal or adult, such as, for example, a horse foal or adult horse, twice daily for three days, preferably, three consecutive days. In a particular embodiment, the twice daily doses are administered to the animal twelve hours apart. The paste formulation is particularly suitable as a product that acts locally in the gut and is minimally absorbed systemically. The paste product specifically addresses the normalization of stool formation and ion and water flow in the intestinal lumen of neonatal, young and adult animals, such as horses, and does not alter gastrointestinal motility, i.e., is not constipating. As but one mode of oral delivery, the paste formulation can be placed in the roof of the animal's mouth. In a particular embodiment, the paste formulation comprises beads (nano or microparticles) comprising enterically coated SB 300 or SP 303 and is orally administered to the animal. In an embodiment, the paste comprising SB 300 enteric beads is orally administered to an animal twice daily for three days. In some embodiments, the paste is orally administered for three consecutive days. In an embodiment, the paste comprising SB 300 enteric beads is orally administered to an animal in need at a dose of 2 mg/kg twice daily for three days. The formulation is especially suitable for the normalization of stool formation in a short time period, e.g., less than a week or less than two weeks; for mitigation of weight loss; and reduction in supportive care costs, rehydration therapies, such as oral rehydration, in a treated animal afflicted with diarrhea.

The types of non-human neonatal, young and adult animals for which the treatment methods are suitable are not particularly limited as to animal type, genus, or species. In general, neonatal, young, or adult farm animals, food-source animals, livestock animals, animals bred or kept for various purposes, such as sport (e.g., racing, riding), transport, domestic, companion, industrial uses (e.g. hauling, pulling, plowing), and the like, are particularly amenable to treatment according to the methods of the invention. For example, encompassed by the methods of the invention is the treatment of Salmonella-infected adult, neonatal or young non-human animals, such as cows (calves), cattle or steer (calves), camels (calves), sheep (lambs), rams, horses (foals), pigs (piglets), goats (kids), bison/buffalo (calves), llamas, donkeys, mules, yaks, etc. Neonatal, young and adult exotic animals, such as zoo animals of various species, are also embraced by the treatments of the invention. In an embodiment, the animals are grazing animals. The treatment of Salmonella-induced diarrhea in neonatal, unweaned and adult animals, for example, cows, steer, calves (bovine, camel, buffalo/bison), sheep, lambs, pigs, piglets, goats and foals (equine) is particularly embraced by the described methods.

In accordance with the described methods, the C. lechleri proanthocyanidin polymer composition reduces chloride flux across intestinal epithelial cells and reduces fluid movement into the intestinal lumen, which results in fluid loss and dehydration associated with secretory diarrhea. Therefore, the pharmaceutically acceptable formulations and methods of the invention are useful in prophylactic and therapeutic applications in the treatment of secretory diarrhea caused by Salmonella spp. infection, especially in preventing the symptoms of dehydration and electrolyte loss that accompany diarrhea, e.g., secretory/watery diarrhea. As noted above, the treatment of diarrhea and its symptoms caused by Salmonella spp. with a proanthocyanidin polymer composition from Croton lechleri, or with a latex, extract or food supplement botanical extract derived therefrom is an unexpected and surprising aspect of the invention, because Salmonella spp. cause diarrhea by a mechanism of action and by affecting cellular pathways and responses that is distinct and different from the mechanism of action associated with the activity of proanthocyanidin polymer compositions.

In an embodiment, young animals treated by the methods of the invention are two to four weeks of age. In an embodiment, the animals are two to four week old calves, e.g., without limitation, bovine or camel calves, having diarrhea caused by infection with Salmonella, or crytosporidia or a combination thereof. In an embodiment, the animals are two to four week old calves, e.g., without limitation, bovine or camel calves, having undifferentiated diarrhea of unknown origin. In other embodiments, the animals treated by the methods of the invention are approximately 3 to 1000 kg in weight; or approximately 5 to 900 kg in weight, or approximately 10 to 350 kg in weight; or approximately 15 to 150 kg in weight; or approximately 25 to 60 kg in weight, or approximately 30 to 50 kg in weight, or approximately 30 to 40 kg in weight. In an particular embodiment, the young animal being treated for diarrhea is a bovine calf of approximately 20 to 40 kg in weight. In an particular embodiment, the young animal being treated for diarrhea is a camel calf of approximately 30 to 50 kg in weight.

In another embodiment, the adult animals treated by the methods of the invention are greater than four months of age. In a related embodiment, the animals have fully developed and competent GI tracts and colon function. In an embodiment, the animal has a weight (mass) of about 400 to 800 kg. In an embodiment, the animal has a weight of about 500 to 700 kg. In an embodiment, the animal has a weight of about 550 to 650 kg. In an embodiment, the animal has a weight of about 600 kg. In an embodiment, the animals have undifferentiated diarrhea of stress-induced origin.

In an embodiment, neonatal and young animals are treated prophylactically with a C. lechleri proanthocyanidin polymer composition, such as SB 300 or SP 303, in enteric or non-enteric form, to prevent or reduce the risk or severity of the debilitating effects of Salmonella-induced diarrheal disease and its associated symptoms, e.g., dehydration and weight loss, in neonatal and young animals. According to the treatment method, a C. lechleri proanthocyanidin polymer composition is administered to neonatal and young animals at a suitable time after birth to protect the animals from diarrhea outbreaks typically caused by infections and adverse environmental conditions. Administering a C. lechleri proanthocyanidin polymer composition to neonatal and young animals can also serve to ameliorate or reduce the risk of the animals' suffering from a more serious or severe form of diarrhea relative to animals that are not provided with the C. lechleri proanthocyanidin polymer composition prior to or during an outbreak of disease or infection. The C. lechleri proanthocyanidin polymer composition can be provided as an enteric or a non-enteric formulation and can be, for example, SB 300 or SP 303. The dose and regimen of C. lechleri proanthocyanidin polymer composition administration are within the skill of the practitioner to determine and will depend on the environmental conditions and health of the neonatal and young animals to be treated. The animals can be prophylactically treated a with C. lechleri proanthocyanidin polymer composition according to the invention, for example and without limitation, one to seven days, one to six days, one to four days, one to three days, or one or two days after birth. The treatment regimen can involve one, two, three, four, five, six, seven or more days, of C. lechleri proanthocyanidin polymer composition administration to the animals, modified or adjusted as necessary or desired, once or multiple times, e.g., twice, three or four times, per day. The animals can be regularly observed and monitored for health improvements and weight gain.

### Physiologically and Pharmaceutically Acceptable Formulations

The C. lechleri proanthocyanidin polymer composition can be provided in any physiologically, pharmaceutically, or therapeutically acceptable form. The pharmaceutically acceptable composition can be formulated for oral administration as, illustratively, but without limitation, powders; crystals; granules; small particles, including particles sized on the order of micrometers, e.g., microspheres and microcapsules; particles sized on the order of millimeters, particles sized on the order of nanometers, e.g., nanoparticles; beads; microbeads; pellets; pills; tablets; microtablets; compressed tablets or tablet triturates; molded tablets or tablet triturates; and in capsules, which are either hard or soft and contain the composition as a powder, particle, bead, solution or suspension. The pharmaceutically acceptable composition can also be formulated for oral administration as a solution or suspension in an aqueous liquid, as a liquid incorporated into a gel capsule, as a gel, as a paste or gel paste, or as any other convenient formulation for administration. The composition can be formulated for rectal administration, as a suppository, enema or other convenient form. The proanthocyanidin polymeric composition can also be provided as a controlled release system (See, e.g., Langer, 1990, Science 249: 1527-1533). The composition can be formulated as a dietary supplement or food supplement, e.g., as described in WO 00/47062, for administration to an animal in need thereof according to the present invention.

The pharmaceutically acceptable formulation can also include any type of pharmaceutically acceptable excipients, additives, carriers, or vehicles. By way of nonlimiting example, diluents or fillers, such as dextrates, dicalcium phosphate, calcium sulfate, lactose, cellulose, kaolin, mannitol, sodium chloride, dry starch, sorbitol, sucrose, inositol, powdered sugar, bentonite, microcrystalline cellulose, or hydroxypropylmethylcellulose can be added to the proanthocyanidin polymer composition to increase the bulk of the composition. In addition, binders, such as, but not limited to, starch, gelatin, sucrose, glucose, dextrose, molasses, lactose, acacia gum, sodium alginate, extract of Irish moss, panwar gum, ghatti gum, mucilage of isapgol husks, carboxymethylcellulose, methylcellulose, polyvinylpyrrolidone, Veegum and starch arabogalactan, polyethylene glycol, ethylcellulose, and waxes, can be added to the formulation to increase its cohesive qualities. Further, lubricants, such as, but not limited to, talc, magnesium stearate, calcium stearate, stearic acid, hydrogenated vegetable oils, polyethylene glycol, sodium benzoate, sodium acetate, sodium chloride, leucine, carbowax, sodium lauryl sulfate and magnesium lauryl sulfate can be added to the formulation. Also, glidants, such as, but not limited to, colloidal silicon dioxide or talc can be added to improve the flow characteristics of a powdered formulation. Disintegrants, such as, but not limited to, starches, clays, celluloses, algins, gums, crosslinked polymers (e.g., croscarmelose, crospovidone, and sodium starch glycolate), Veegum, methylcellulose, agar, bentonite, cellulose and wood products, natural sponge, cation-exchange resins, alginic acid, guar gum, citrus pulp, carboxymethylcellulose, or sodium lauryl sulfate with starch can also be added to facilitate disintegration of the formulation in the intestine.

In some embodiments, the pharmaceutically acceptable formulations contain the proanthocyanidin polymer composition with an enteric coating, in addition to another pharmaceutically acceptable vehicle. In an embodiment, the proanthocyanidin polymer composition can be directly-compressed into a tablet. The tablet can be without excipients and of pharmaceutically acceptable hardness and friability, optionally, with a lubricant, e.g., without limitation, magnesium stearate, and enteric coated. In another embodiment, the pharmaceutically acceptable compositions containing the proanthocyanidin polymer composition alternatively include one or more substances that either neutralize stomach acid and/or enzymes or are active to prevent secretion of stomach acid. These formulations can be prepared by methods known in the art (See, e.g., methods described in Remington's “The Science and Practice of Pharmacy,” 22nd Edition, Editor-in-Chief: Lloyd V. Allen, Jr., Pharmaceutically acceptable Press, Royal Pharmaceutically acceptable Society, London, UK, 2013; and U.S. Pat. No. 7,323,195).

In an embodiment, the proanthocyanidin polymer composition is formulated with a substance that protects the proanthocyanidin polymer and/or the polymer composition from the stomach environment. For such protection, the proanthocyanidin polymer composition can be enteric coated. Enteric coatings are those coatings that remain intact in the stomach, but will dissolve and release the contents of the dosage form once it reaches the small intestine. A large number of enteric coatings are prepared with ingredients that have acidic groups such that, at the very low pH present in the stomach, i.e. pH 1.5 to 2.5, the acidic groups are not ionized and the coating remains in an undissociated, insoluble form. At higher pH levels, such as in the environment of the intestine, the enteric coating is converted to an ionized form, which can be dissolved to release the proanthocyanidin polymer composition. Other enteric coatings remain intact until they are degraded by enzymes in the small intestine, and others break apart after a defined exposure to moisture, such that the coatings remain intact until after passage into the small intestines. A variety of polymers are useful for the preparation of enteric coatings, and the application of an enteric coating to the proanthocyanidin polymer composition can be accomplished by any method known in the art for applying enteric coatings, as may be found, for example, and without limitation, in U.S. Pat. Nos. 7,323,195 and 7,341,744, incorporated herein by reference.

In another embodiment, the pharmaceutically acceptable composition of the proanthocyanidin polymer composition is formulated as enteric coated granules or powder (microspheres with a diameter of 300-500 microns) provided in either hard shell gelatin capsules or suspended in an oral solution for pediatric administration. The enteric coated proanthocyanidin polymer composition powder or granules can also be mixed with food, particularly for administration to neonatal or young animals. Such preparations may be prepared using techniques well known in the art. In addition, the proanthocyanidin polymer composition granules and powder can be prepared using any method known in the art, such as, but not limited to, crystallization, spray-drying or any method of comminution, preferably using a high speed mixer/granulator, as described, for example and without limitation, in U.S. Pat. No. 7,323,195, incorporated herein by reference.

In other embodiments, the proanthocyanidin polymer composition is in the form of an aqueous suspension in admixture with suitable excipients. Non-limiting examples of excipients that are suitable for the manufacture of aqueous suspension include suspending agents, for example, methylcellulose, sodium carboxymethylcellulose, hydroxy-propylmethylcellulose, sodium alginate, polyvinyl-pyrrolidone, gum tragacanth and gum acacia; dispersing or wetting agents, which may be a naturally-occurring phosphatide, e.g., lecithin, or condensation products of an alkylene oxide with fatty acids, e.g., polyoxyethylene stearate, or condensation products of ethylene oxide with long chain aliphatic alcohols, e.g., heptadecaethyleneoxycetanol, or condensation products of ethylene oxide with partial esters derived from fatty acids and a hexitol, for example, polyoxyethylene sorbitol monooleate, or condensation products of ethylene oxide with partial esters derived from fatty acids and hexitol anhydrides, such as polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, e.g., sucrose, saccharin or aspartame.

Dispersible powders and granules suitable for the preparation of an aqueous suspension by the addition of water provide the proanthocyanidin polymer composition in admixture with a dispersing or wetting agent, suspending agent and one or more preservatives. Suitable dispersing or wetting agents and suspending agents are exemplified by those stated above. Additional excipients, for example, sweetening, flavoring and coloring agents, may also be present.

In an embodiment, the proanthocyanidin polymer composition is a gel or gel formulation. In an embodiment, the proanthocyanidin polymer composition is a paste formulation. In an embodiment, the paste formulation contains a purified botanical extract derived from C. lechleri. In another embodiment, the paste formulation contains enterically coated beads comprising SB 300 or SP 303. In an embodiment, the paste formulation contains enteric protected SB 300 beads. In an embodiment, the gel or paste is contained or preloaded in a delivery device, such as a syringe, e.g., a needle-less syringe, or other type of applicator or delivery system, especially for oral delivery. A gel or paste formulation is particularly suited for administration to Salmonella spp.-infected neonatal and young foals, but also is applicable for other Salmonella spp.-infected adult and neonatal animals, such as those described herein. In an embodiment, the gel or paste is not contained in a delivery device, but is administered to the roof of the mouth of the animal, particularly one that is too incapacitated or ill to eat or drink, thereby eschewing an oral or other mode of administration. In an embodiment, the gel comprises pH-sensitive polymeric particles, such as microparticles or nanoparticles, to allow for pH-dependent uptake of the active compound into cells and/or the pH-dependent release of the active compound in different pH environments in an animal. Processes for generating granules and particles comprising the proanthocyanidin polymer composition or a compressible form thereof are as known and practiced in the art, and as provided, for example, in U.S. Pat. No. 7,341,744, the contents of which are incorporated by reference herein. In an embodiment, gels are prepared for oral delivery and contain copolymers, such as poloxamers and Pluronics of different types, e.g., Pluronic F.

In another embodiment, the proanthocyanidin polymer composition is in a paste formulation, preferably for oral administration. For example, an oral paste may comprise, without limitation, an oily vehicle or excipient, such as a hydrophobic oily vehicle, a basifying agent, a flavoring agent and a coloring agent. Illustrative and nonlimiting examples of hydrophobic oily vehicles include vegetable oil, triglyceride or polypropylene glycol, as well as a thickening agent, e.g., aluminum stearate. Flavoring agents can include, for example, fruit flavors, mint flavors, honey flavor, and other natural and organic flavorings known to those skilled in the art. Coloring agents can include, for example, iron oxide or titanium dioxide. Alternatively, the oily vehicle can be liquid paraffin or other suitable waxes, including a thickening agent. In an embodiment, the paste formulation contains beads with enterically coated SB 300 or SP 303, which is administered to an animal, such as, for example, a foal or adult horse, at a dose of 2 mg/kg. More particularly, the paste formulation containing enterically coated SB 300 beads is administered to the animal at a dose of 2 mg/kg, twice a day for three days. In an embodiment, the paste containing enteric protected SB 300 beads is administered twice a day at twelve hour intervals.

Oily suspensions may be formulated by suspending the C. lechleri proanthocyanidin polymer as active ingredient in a vegetable oil, e.g., arachis oil, olive oil, sesame oil or coconut oil, or in mineral oil, such as liquid paraffin. The oily suspensions may contain a thickening agent, e.g., beeswax, hard paraffin or cetyl alcohol. Oral preparations can include sweetening agents as mentioned above and flavoring agents to improve palatability. Pharmaceutically acceptable preservatives, for example, an anti-oxidant such as ascorbic acid, can also be added to such compositions.

The C. lechleri proanthocyanidin polymer pharmaceutical compositions used in the methods of the invention may also be in the form of an oil-in-water emulsions. The oily phase may be a vegetable oil such as olive oil or arachis oil, or a mineral oil such as liquid paraffin or mixtures of these oils. Examples of emulsifying agents include, without limitation, naturally-occurring phosphatides, e.g., soy bean, lecithin, and esters or partial esters derived from fatty acids and hexitol anhydrides, e.g., sorbitan monooleate, and condensation products of partial esters with ethylene oxide, e.g., polyoxyethylene sorbitan monooleate. Sweetening, coloring and flavoring agents can be included in the emulsions.

Syrups and elixirs containing the C. lechleri proanthocyanidin polymer may also can be formulated with sweetening agents, for example, glycerol, propylene glycol, sorbitol or sucrose. Such formulations may also contain a demulcent, a preservative and flavoring and coloring agents. The pharmaceutical compositions may be in the form of a sterile, orally deliverable or administrable aqueous or oleagenous suspension. This suspension may be formulated according to methods known in the art using suitable dispersing or wetting agents and suspending agents, such as those mentioned above. The sterile pharmaceutical preparation may also be a sterile solution or suspension in a non-toxic parenterally-acceptable diluent or solvent, for example, a solution in 1,3-butane diol. Illustrative, acceptable vehicles and solvents that may be used in the preparations include water, Ringer's solution and isotonic sodium chloride solution. Co-solvents, e.g., ethanol, propylene glycol or polyethylene glycols, may also be included. In addition, sterile, fixed oils, e.g., any bland, fixed oil such as synthetic mono- or diglycerides, are conventionally employed as solvents or suspending media and may be used. In addition, fatty acids, such as oleic acid and the like, may be used in injectable preparations.

### Dosage Forms and Administration

In a particular embodiment for treating diarrhea in neonatal animals, e.g., without limitation, bovine and camel calves, foals, kids, lambs, etc., the proanthocyanidin polymer composition is in powder, e.g., reconstitutable powder, form. The composition may be enterically coated or not enterically coated. In an embodiment, the neonates are less than one week in age. In an embodiment, the neonatal animals are bovine calves or camel calves.

In a particular embodiment for treating diarrhea in adult animals, e.g., without limitation, horses, camels, llamas, cows, sheep, bison, etc., the proanthocyanidin polymer composition is in powder, e.g., reconstitutable powder, form. The composition may be enterically coated or not enterically coated. In an embodiment, the non-human adult animal is of an age that its large intestine/bowel and colon are fully competent and functional. In an embodiment, the non-human animal is greater than four months of age. In an embodiment, the non-human animals are adult horses or camels. In an embodiment, the animals are afflicted with Salmonella-induced diarrhea. In an embodiment, in addition to Salmonella-induced diarrhea, the animal experiences involvement of one or more of a bacterial, e.g., Clostridium spp. infection, a viral infection, e.g., rotavirus and/or coronavirus, whose mechanism of action involves infection and subsequent destruction of the cells lining the intestinal tract, or a parasitic infection, e.g., nematodes.

In an embodiment, the powder form of the proanthocyanidin polymer composition used for treatment is reconstituted or mixed with liquid, such as oral electrolytes, milk or a milk replacer, water, physiological saline, to produce a liquid form or suspension. Milk replacer is generally a source of protein from different origins (for example, milk from a different species, soy, or eggs) and energy (lactose and fat) given to the calf or other animals to replace milk from the mother. In a specific embodiment, the proanthocyanidin polymer composition is mixed at 200 mg to 800 mg per kg of the powder milk replacer prior to reconstitution. In an embodiment, the powder form of the proanthocyanidin polymer composition is provided in the form of individual dosages in packets, e.g., packaged dosage forms, wherein some number of individual packets are provided for use in a treatment regimen. In certain embodiments, the packaged dosage form contain 50 to 600 mg of the proanthocyanidin polymer composition, preferably, 200 to 300 mg of the proanthocyanidin polymer composition. The number of individual doses that can be packaged and provided together is not intended to be limiting, and can include, for example, one to twenty packaged doses; one to ten packaged doses; two, four, six, eight, ten, or more packaged doses, as well as numbers of packaged doses in-between the foregoing, for efficiency of use, handling and for commercial efficacy. Those skilled in the art will appreciate that due to the higher purity of compositions such as SP-303 or crofelemer and SB-300, more by weight of SB-300 than SP-303 will need to be used in formulations to achieve the same amount of the active ingredient of the proanthocyanidin polymer composition. SB-300 generally has about 67% by weight of the proanthocyanidin polymer composition while SP-303 has higher purity, for example 99-100%.

In another embodiment, the powder form of the proanthocyanidin polymer composition is provided in a container, such as a bag, box, bucket, or pail (e.g., 5 lb. to 25 lb. pails), in which the powder can be in an amount of, for example, 100 grams (g) or more, and can optionally include a measuring device, such as a scoop, cup, spoon, trowel, dipper, or ladle. Such containers encompass, for example, an individual daily dose of the proanthocyanidin polymer composition; or an amount suitable for multiple treatments, e.g., a two-day treatment, three-day treatment, four day treatment, etc. An effective amount of the powder can also be mixed with feed for consumption by the young animals, e.g., calves, in need thereof. In certain embodiments the proanthocyanidin polymer composition is administered in an amount of 1 to 8 mg/kg twice a day for 3 to 5 days.

In an embodiment, the C. lechleri proanthocyanidin polymer composition is administered or delivered to a neonatal or adult animal afflicted with diarrhea and in need thereof by providing the compound as a bolus or pill. In an embodiment, the proanthocyanidin polymer composition formulated as bolus, i.e., a pill, capsule, or tablet, is orally administered to the animals afflicted with diarrhea or symptoms thereof directly in the mouth. In a particular embodiment, the treatment regimen comprises administering a dose of 250 mg of the product, e.g., as embraced by one bolus per sick animal for a determined time period, for example, for one, two, or three or more days. The product can be provided to an animal in need thereof in portions of the complete dose, in which the portions are administered one or two or more times per day. Alternatively, the complete dose can be administered to an animal in need thereof one or two or more times per day. In a particular embodiment, the treatment encompasses a dose of 250 mg given two times a day. In another embodiment, the treatment encompasses an oral bolus dose of 250 mg given two times a day for 3 days. In an embodiment, the dose is the C. lechleri proanthocyanidin polymer composition, SB 300, in enteric form or in non-enteric form, e.g., a reconstituted powder form.

The routes of administration of the C. lechleri proanthocyanidin polymer product to afflicted animals are not intended to be limiting. Illustratively, administration can be via any suitable, convenient or preferred route of administration including oral, buccal, dental, periodontal, via food source (animal feed), nutrition source, or libation source, otic, inhalation, endocervical, intramuscular, subcutaneous, intradermal, intracranial, intralymphatic, intraocular, intraperitoneal, intrapleural, intrathecal, intratracheal, intrauterine, intravascular, intravenous, intravesical, intranasal, ophthalmic, biliary perfusion, cardiac perfusion, spinal, sublingual, topical, transdermal, intravaginal, rectal, ureteral, or urethral. In certain embodiments, oral, buccal, and food and/or drink supplement are particularly suitable routes. In an embodiment, the product is an aqueous formulation and is provided to the animal as a drench or directly from a ready-to-use (RTU) bottle directed to the esophageal cavity so as to more effectively reach the animal's intestine/gut for optimal activity. In a related embodiment, administration can also be by inclusion in the regular or special diet of the animal, such as in a functional food for the animals or companion animals.

Dosage forms can include, without limitation, oral, injectable, transdermal, aerosol including metered aerosol, chewable products or pellets, capsules, capsule containing coated particles, nanoparticles, or pellets, capsule containing delayed release particles, capsule containing extended release particles, concentrates, creams and augmented creams, suppository creams, discs, dressings, elixirs, emulsions, enemas, extended release films or fibers, gases, gels, metered gels, granules, delayed release granules, effervescent granules, implants, inhalants, injectable lipid complexes, injectable liposomes, inserts or devices, extended release inserts, intrauterine devices, jellys, liquids, extended release liquids, lotions, augmented lotions, oils, ointments, augmented ointments, pastes, pastilles, pellets, powders, reconstituted powders, extended release powders, metered powders, solutions, drops, concentrated solutions, gel forming solutions/drops, sponges, sprays, metered sprays, suppositories, suspensions, suspensions/drops, extended release suspensions, syrups, tablets/pills, chewable tablets/pills, tablets/pills containing coated particles, delayed release tablets/pills, dispersible tablets/pills, effervescent tablets/pills, extended release tablets/pills, orally disintegrating tablets/pills, tapes, or troches/lozenges. The dosages can be provided as formulations, compositions, pharmaceutically acceptable formulations and compositions, physiologically acceptable formulations and compositions, including pharmaceutically and physiologically acceptable carrier, excipients, diluents, or vehicles as known and used in the art.

For oral administration, the C. lechleri proanthocyanidin polymer product, or a composition thereof, is preferably encapsulated and formulated with suitable carriers, and the like, in solid dosage forms. Nonlimiting examples of suitable carriers, excipients, diluents and vehicles include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, edible oils, and the like. The formulations can also include lubricating agents, wetting agents, emulsifying and suspending agents, preserving agents, sweetening agents or flavoring agents. The compositions can be formulated to provide rapid, sustained, extended, or delayed release of the active ingredient after administration to the animal by employing protocols and methods well known in the art. The formulations can also include compounds or substances that reduce proteolytic degradation and promote absorption such as, for example, surface active agents.

As will be appreciated by those having skill in the art, the specific dose can be calculated according to the approximate body weight, body mass, or body surface area of the animal, or the volume of body space or mass to be occupied. The dose also depends on the particular route of administration selected by the practitioner. Further refinement of the calculations necessary to determine an appropriate dosage for treatment is routinely made by those of ordinary skill in the art, for example, using appropriate assays and analytical procedures, such as has been described for certain compounds (e.g., Howitz et al., Nature, 425:191-196, 2003). Exact dosages can be determined based on standard dose-response studies. Therapeutically effective doses for treatment of afflicted animals can be determined, by titrating the amount of the active product given to the animal to arrive at the desired therapeutic effect, while minimizing side effects.

For use in treating Salmonella-induced diarrhea and its symptoms in neonatal and young animals in accordance with the methods of the invention, a therapeutically acceptable form of the C. lechleri proanthocyanidin polymer composition is administered, particularly orally administered, in an amount ranging from 0.1 to 100 mg/kg per day, once, twice or more daily. In other embodiments, the amount can range from about 0.1 to about 10 mg/kg/day, once, twice or more daily; or from about 0.1 to about 25 mg/kg/day, once, twice or more daily; or from about 0.1 to about 30 mg/kg/day, once, twice or more daily; or from about 0.1 to about 40 mg/kg/day, once, twice or more daily. In other embodiments, the dose can be 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, etc., as well as incremental dose amounts in between. In still other embodiments, the amount can range from about 1 to about 10 mg/kg/day once, twice or more daily; or from about 1 to about 5 mg/kg/day, from about 1 to about 8 mg/kg/day, from about 1 to about 10 mg/kg/day, or from about 2 to about 4 mg/kg/day once, twice or more daily. In other embodiments, the foregoing amounts of the C. lechleri proanthocyanidin polymer composition are administered, for example, twice daily, three times daily, four times daily, or more than four times daily, rather than once per day. Higher doses, e.g., 50 mg/kg or 100 mg/kg per day or twice or more daily, may be required, as necessary, to treat diarrhea and accompanying dehydration in the neonatal and young animals.

For use in treating Salmonella-induced diarrhea and its symptoms in adult animals in accordance with the methods of the invention, a therapeutically acceptable form of the C. lechleri proanthocyanidin polymer, including a C. lechleri botanical extract, is administered, particularly orally administered, in an amount ranging from 0.1 to 100 mg/kg per day. In other embodiments, the amount can range from about 0.1 to about 10 mg/kg/day; or from about 0.1 to about 25 mg/kg/day, or from about 0.1 to about 30 mg/kg/day, or from about 0.1 to about 40 mg/kg/day. In an embodiment, the dose is 2 mg/kg twice a day. In an embodiment, the 2 mg/kg dose is administered twice a day for three days. Higher doses, e.g., 50 mg/kg/day, 100 mg/kg/day, 200 mg/kg/day, or greater, may be required, as necessary, to treat the diarrhea and accompanying dehydration in adult animals. In other embodiments, the dose can be 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, etc., as well as incremental dose amounts in between.

In other embodiments, for the treatment methods, a suitable dose for the C. lechleri proanthocyanidin polymer product, or the C. lechleri proanthocyanidin polymer composition, such as SP 303 or SB 300, may range from about 1 mg to about 1000 mg, either daily or multiple times per day. In an embodiment, a suitable dose may range from about 10 mg to about 500 mg, either daily or multiple times per day. In an embodiment, a suitable dose may range from about 50 mg to about 350 mg, either daily or multiple times per day. In an embodiment, a suitable dose may range from about 30 mg to about 400 mg, either daily or multiple times per day. In an embodiment, a suitable dose may range from about 100 mg to about 250 mg, either daily or multiple times per day. In an embodiment, a suitable dose may range from about 50 mg to about 300 mg, either daily or multiple times per day. It will be understood that the ranges include the lower and higher amounts specified, as well as amounts in between. The doses administered multiple times per day can be given for consecutive days, e.g., two days, three days, four days, five days, six, days, seven days, or more, in some embodiments. A dose administered multiple times per day may embrace two, three, four, five, six, or more times per day. Other dosing schedules, such as every other day, or every third day, every fourth day, etc. are embraced by the invention. In addition, one having skill in the art will appreciate that doses and amounts administered to the animal can vary, given the wide range of weights of the animals undergoing treatment, as well as the animal species and type of digestive system, e.g., ruminant or non-ruminant. In an embodiment the C. lechleri proanthocyanidin polymer is SB 300. In an embodiment the C. lechleri proanthocyanidin polymer is enterically coated SB 300. In an embodiment the C. lechleri proanthocyanidin polymer is non-enterically coated SB 300.

In some embodiments, daily doses, including multiple daily doses, e.g., twice or three times a day, of the C. lechleri proanthocyanidin polymer product may be 2 mg, 5 mg, 10 mg, 15 mg, 20 mg, 25 mg, 50 mg., 100 mg, 150 mg, 175 mg, 200 mg, 225 mg, 250 mg, 275 mg, 300 mg, 325 mg, 350 mg, 375 mg, 400 mg, 500 mg (or there between) per animal. Administration schedules may also be altered to achieve a therapeutically effective concentration of the C. lechleri proanthocyanidin polymer to treat the diarrhea and its symptoms as described herein. By way of specific, yet nonlimiting example, a suitable dosage amount for use in the methods according to the invention is 250 mg administered once or twice daily. In some embodiments, the compound may be administered once per day, twice per day, thrice per day, 4 times per day, 5 times per day, 7 times per day or 10 times per day. Often the dosage is divided into equal parts administered throughout the day, however in some embodiments related to treating more severe or entrenched symptoms, it may be useful to tailor the dosage administration schedule so that most of the daily treatment is administered at a predetermined time of the day, e.g., the beginning half of the day. In some embodiments, about 50% 60%, 70% or 80% of the dosage is administered in the first half of the day. In other embodiments, it may be more appropriate to administer most of the dosage in the latter half of the day so that about 50%, 60%, 70% or 80% of the dosage is administered in the latter half of the day.

It will be understood that the dose amount actually administered can be determined by the practitioner, in the light of the relevant circumstances, including the severity of the disease, condition, or symptoms thereof being treated, the form of the product to be administered, the age, weight, and response of the individual animal receiving treatment, as well as the chosen route of administration.

The methods of the invention further embrace the administration of pharmaceutically acceptable formulations of the proanthocyanidin polymer composition either alone or in combination with other supplements or agents for treatment or amelioration of the symptoms of secretory diarrhea, such as rehydration agents, electrolytes (e.g., sodium, potassium, magnesium, chloride and formulations thereof), antibiotics, gut-lining protectants, such as kaolin, pectin, or bismuth liquid, and fluid adsorbents, such as attapulgite. Other agents may include anti-motility agents, although because many of the microorganisms and pathogens that are associated with diarrhea induction in neonatal and young animals concomitantly decrease gut motility, the use of anti-motility drugs may be contraindicated. Natural biological products, e.g., Lactobacillus, Bifidobacterium, or Streptococcus faecium, other bacteria and yeast microorganisms, or probiotics, may also be employed as additives to restore the natural balance of intestinal flora in the affected neonatal animals. Such natural biological products, e.g., probiotics as known in the art, may be administered in conjunction with the C. lechleri proanthocyanidin polymer or composition thereof, for example, prior to, at the same time as, or after the administration of the proanthocyanidin polymer or composition to a non-human animal. In addition, a reconstituted C. lechleri proanthocyanidin polymer or composition thereof may include probiotics in accordance with the present invention.

### Exemplary Specific Embodiments Encompassed by the Invention

The present invention is further directed to uses and methods encompassed by the following embodiments.

The present invention is further directed to the use of an aqueous soluble proanthocyanidin polymer from Croton lechleri in treating diarrhea resulting from Salmonella spp. infection of a non-human animal, the method comprising orally administering to the non-human animal in need thereof an effective amount of the Croton lechleri proanthocyanidin polymer to treat the Salmonella-induced diarrhea in the non-human animal. In an embodiment, the C. lechleri proanthocyanidin polymer is administered as an enteric coated pharmaceutical composition or as a non-enteric pharmaceutical composition. In an embodiment, the non-human animal is a juvenile animal or an adult animal, such as a bovine, equine, camel, ovine, swine, or goat. In more particular embodiments, the non-human animal is an equine animal, a bovine animal, or a camel. In an embodiment, the C. lechleri proanthocyanidin polymer is administered in an amount of 250 mg twice daily. In an embodiment, the non-human animal is infected with one or more of bacteria that are not a Salmonella spp.; parasites; viruses; or protozoa. In an embodiment, the C. lechleri proanthocyanidin polymer is administered as powder reconstituted with a liquid selected from oral electrolytes, milk, milk replacer or substitute, physiological saline, or water. In an embodiment, the C. lechleri proanthocyanidin polymer is administered as a bolus. In an embodiment, the C. lechleri proanthocyanidin polymer is administered in animal feed or drink. In an embodiment, the C. lechleri proanthocyanidin polymer is formulated in the form of a gel, paste, or gel paste, which may be administered to the animal by topical application to the roof of the animal's mouth. In an embodiment, the gel, paste, or gel paste is contained in a delivery device, which may be a syringe. In an embodiment, the gel, paste, or gel paste comprises polymeric microparticles or nanoparticles containing the C. lechleri proanthocyanidin polymer. In an embodiment, the polymeric microparticles or nanoparticles are pH-sensitive. In an embodiment, the C. lechleri proanthocyanidin polymer is administered to the animal in an amount of at least 50 mg to 250 mg. In an embodiment, the symptoms associated with the Salmonella-induced diarrhea in the non-human animal include dehydration and electrolyte loss. In an embodiment, the C. lechleri proanthocyanidin polymer composition is selected from the group consisting of SB 300, SP 303 and crofelemer. In a particular embodiment, the C. lechleri proanthocyanidin polymer composition is SB 300. In an embodiment, the paste comprises enteric coated SB 300 and is administered to the animal in an amount of 2 mg/kg twice a day for three days. In an embodiment, the paste is administered twice a day, twelve hours apart.

In another embodiment, the invention encompasses a medicament comprising an aqueous soluble proanthocyanidin polymer from Croton lechleri for use in treating a diarrhea resulting from Salmonella spp. infection of a non-human animal, the method comprising orally administering to the non-human animal in need thereof an effective amount of the Croton lechleri proanthocyanidin polymer to treat the Salmonella-induced diarrhea in the non-human animal. In a related embodiment, the invention encompasses the use of an aqueous soluble proanthocyanidin polymer from Croton lechleri in the manufacture of a medicament for treating diarrhea resulting from Salmonella spp. infection of a non-human animal, the method comprising orally administering to the non-human animal in need thereof an effective amount of the Croton lechleri proanthocyanidin polymer to treat the Salmonella-induced diarrhea in the non-human animal.

The following examples are representative of the present invention in its various aspects and are not intended to be limiting.

### EXAMPLES

### Example 1

Evaluation of the Effect of Oral Administration of a Croton lechleri Proanthocyanidin Polymer Composition on the Fecal Scores of Salmonella typhimurium-Infected Neonatal Bovine Calves Afflicted with Diarrhea

A small completely randomized study was conducted to evaluate the effect of oral administration of 250 mg of a Croton lechleri proanthocyanidin polymer composition, i.e., oral SB 300, on fecal consistency of bovine calves infected with Salmonella typhimurium and receiving treatment twice daily for 3 consecutive days. Fecal consistency scores were determined throughout the treatment period. A total of 82 calves were randomly allocated into one of two treatment groups; 39 calves were allocated into the control groups and 43 calves were allocated into the treatment groups. All calves were clinically affected with diarrhea induced by Salmonella infection and received palliative therapy according with the farm standard operating procedures. In addition, calves allocated the treatment group received the same palliative care and were treated orally with the Croton lechleri proanthocyanidin polymer SB 300 composition, as above.

Calves were scored for fecal consistency using a three level score system; 0=solid/normal well-formed feces, 1=pasty feces, and 2=watery diarrhea. Each calf received a total of 6 fecal scorings (twice daily, morning and afternoon) for three days following the diarrhea diagnosis. Data were analyzed using repeated measures ANOVA.

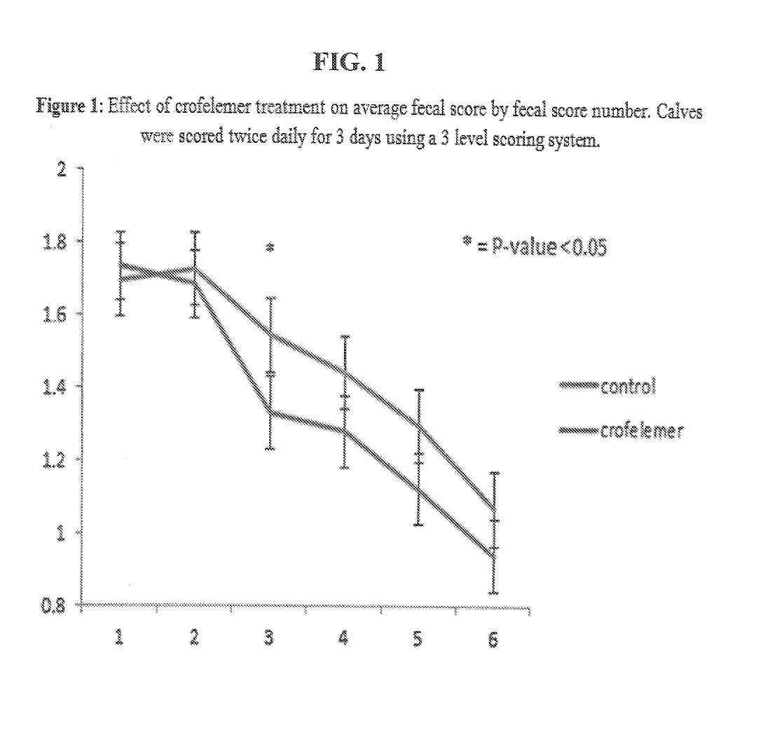

Treatment with the Croton lechleri proanthocyanidin polymer extract composition (SB 300) had a strong tendency to improve (i.e., decrease) fecal scores (P value=0.05). Overall, the average fecal score for control calves was 1.46 and for the treatment calves it was 1.34 (P value=0.05). Fecal consistency scores were similar between treatment group at the beginning of the study. Calves treated with the Croton lechleri proanthocyanidin polymer extract composition demonstrated faster improvement on diarrhea scores, starting on the second day of treatment (See, FIG. 1). The study results demonstrate that calves having malabsorption-type diarrhea resulting from infection with Salmonella bacteria showed improvement in fecal scores, indicating the successful treatment of diarrhea, when treated with the SB 300 composition.

### Example 2

A representative paste composition of the present invention, which comprises enterically coated SB 300 beads, is presented in this Example. For administration to animals and as noted hereinabove, the paste containing enteric SB 300 beads may be contained in a syringe. A paste containing enteric coated SB 300 beads may contain the following components:

| Component | % w/w | Theoretical mg/syringe |
|---|---|---|
| SB 300 enteric beads | 21.91 | 3286.6* |
| Vegetable oil | 64.42 | 9663.5 |
| Cetyl alcohol | 9.76 | 1464.2 |
| Apple flavor | 0.08 | 11.7 |
| Silicon dioxide | 2.73 | 410.0 |
| Butylated hydroxytoluene | 0.04 | 5.9 |
| Titanium dioxide | 1.05 | 158.1 |
| Total | 100.0 | 15000 |

*3286.6 mg SB 300 enteric beads corresponds to 880 mg theoretical SB 300.

All patents, patent applications and publications referred to or cited herein are hereby incorporated by reference in their entireties for all purposes.

It is understood that the embodiments and examples described herein are for illustrative purposes and that various modifications or changes in light thereof will be suggested to persons skilled in the pertinent art and are to be included within the spirit and purview of this application and scope of the appended claims. It is to be understood that suitable methods and materials are described herein for the practice of the embodiments; however, methods and materials that are similar or equivalent to those described herein can be used in the practice or testing of the invention and described embodiments.

What is claimed is:

1. A method of treating diarrhea resulting from Salmonella spp. infection of a non-human animal, the method comprising orally administering to the non-human animal in need thereof an aqueous soluble proanthocyanidin polymer from Croton lechleri in an amount effective to treat the Salmonella-induced diarrhea in the non-human animal.

2. The method according to claim 1, wherein the C. lechleri proanthocyanidin polymer is administered as an enteric coated pharmaceutical composition.

3. The method according to claim 1, wherein the C. lechleri proanthocyanidin polymer is administered as a non-enteric pharmaceutical composition.

4. The method according to any one of claims 1 to 3, wherein the non-human animal is a juvenile animal.

5. The method according to any one of claims 1 to 3, wherein the non-human animal is an adult animal.

6. The method according to any of claims 1 to 5, wherein the non-human animal is selected from bovine, equine, camel, ovine, swine, or goats.

7. The method according to claim 6, wherein the non-human animal is an equine animal.

8. The method according to claim 6, wherein the non-human animal is a bovine animal.

9. The method according to claim 6, wherein the non-human animal is a camel.

10. The method according to any one of claims 1 to 9, wherein the C. lechleri proanthocyanidin polymer is administered in an amount of 250 mg twice daily.

11. The method according to any one of claims 1 to 10, wherein the non-human animal is infected with one or more of bacteria that are not a Salmonella spp., parasites, viruses, or protozoa.

12. The method according to any one of claims 1 to 11, wherein the C. lechleri proanthocyanidin polymer is administered as powder reconstituted with a liquid selected from oral electrolytes, milk, milk replacer, physiological saline, or water.

13. The method according to any one of claims 1 to 12, wherein the C. lechleri proanthocyanidin polymer is administered as a bolus.

14. The method according to any one of claims 1 to 12, wherein the C. lechleri proanthocyanidin polymer is administered in animal feed or drink.

15. The method according to any one of claims 1 to 11, wherein the C. lechleri proanthocyanidin polymer is formulated in the form of a gel, paste, or gel paste.

16. The method according to claim 15, wherein the gel, paste, or gel paste is administered to the animal by topical application to the roof of the animal's mouth.

17. The method according to claim 15 or claim 16, wherein the gel, paste, or gel paste is contained in a delivery device.

18. The method according to claim 17, wherein the delivery device is a syringe.

19. The method according to any one of claims 15 to 18, wherein the gel, paste, or gel paste comprises polymeric microparticles or nanoparticles containing the C. lechleri proanthocyanidin polymer.

20. The method according to claim 19, wherein the polymeric microparticles or nanoparticles are pH-sensitive.

21. The method according to any one of claims 1 to 20, wherein the C. lechleri proanthocyanidin polymer is administered to the animal in an amount of at least 50 mg to 250 mg.

22. The method according to any one of claims 1 to 21, wherein the symptoms associated with the Salmonella-induced diarrhea in the non-human animal include dehydration and electrolyte loss.

23. The method according to any one of claims 1 to 22, wherein the C. lechleri proanthocyanidin polymer composition is selected from the group consisting of SB 300, SP 303 and crofelemer.

24. The method according to claim 23, wherein the C. lechleri proanthocyanidin polymer composition is SB 300.

25. The method according to claim 20, wherein the paste comprises enteric coated SB 300 and is administered to the animal in an amount of 2 mg/kg twice a day for three days.

26. The method according to claim 25, wherein the paste is administered twice a day, twelve hours apart.

27. The method according to any one of claims 1 to 26, wherein the C. lechleri proanthocyanidin polymer or composition thereof is administered in conjunction with probiotics.
